(19) United States
(12) Patent Application Publication
(10) Pub. No.: US 2006/0216835 A1
(43) Pub. Date: Sep. 28, 2006

(54) Method of separating unattached Raman-active tag from bioassay or other reaction mixture

(75) Inventor: Frank John Mondello, Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/257,165

(22) Filed: Oct. 24, 2005

Related U.S. Application Data
(63) Continuation-in-part of application No. 11/087,419, filed on Mar. 24, 2005.

(51) Int. Cl. G01N 33/551

(52) U.S. Cl. 436/524; 977/900

(57) ABSTRACT

A superparamagnetic Raman-active complex that includes a Raman-active tag, a target, and a superparamagnetic particle is disclosed. A method of applying a magnetic field to a mixture is also disclosed. The mixture includes a Raman-active tag unattached to a target and a superparamagnetic Raman-active complex. Also disclosed is a method of separating a Raman-active tag unattached to a target from a Raman-active complex. The Raman-active complex includes a Raman-active tag attached to a target.

### CROSS REFERENCE TO RELATED APPLICATIONS

This application is a continuation-in-part of U.S. patent application Ser. No. 11/087,419, filed Mar. 24, 2005, which is hereby incorporated by reference.

### BACKGROUND

The invention includes embodiments that may relate to bioassays or other reaction mixture. The invention includes embodiments that may relate to a method of separating an unattached tag from a bioassay or other reaction mixture.

### DESCRIPTION OF RELATED ART

Raman-active tags 100 may detect the presence of pathogenic organisms or other materials to or against which the Raman-active tags are directed. FIG. 1 is a schematic representation of a Raman-active tag 100 that includes a Raman-active particle 110 attached to one or more target-binding moieties 112. The target-binding moiety 112 on the Raman-active tag 100 is attached to one or more targets 212 to form a Raman-active complex 200, as shown in FIG. 2A and FIG. 2B. FIG. 2A and FIG. 2B are schematic representations of a Raman-active complex 200 having a Raman-active tag 100 and a target 212. Detection of the target 212 is based on the presence of a Raman signal after removing any Raman-active tags 100 that are unattached to a target 212, from a test mixture. Failure to minimize or eliminate unattached Raman-active tags 100 may result in a false positive detection of the presence of the target 212. Centrifugation is a method used to separate unattached Raman-active tags 100 from Raman-active complexes 200 that are attached to a target; however, centrifugation may be undesirable because the Raman-active tags 100 have a density such that the Raman-active tags 100 pellet together with the Raman-active complexes 200 and targets 212.

Thus, it may be desirable to have a method of separating unattached Raman-active tag from bioassay or other reaction mixture that differs from currently available methods.

### BRIEF DESCRIPTION

An embodiment of the invention provides a superparamagnetic Raman-active complex. The superparamagnetic Raman-active complex includes a Raman-active tag attached to a target and a superparamagnetic particle. The superparamagnetic particle is attached to the target or the Raman-active tag.

Another embodiment provides a method of applying a magnetic field to a mixture. The mixture includes at least one Raman-active tag unattached to a target and at least one superparamagnetic Raman-active complex. The Raman-active complex includes a Raman-active tag attached to a target.

Another embodiment provides a method of separating a Raman-active tag unattached to a target from a Raman-active complex. The method includes applying a magnetic field to a mixture. The mixture includes at least one Raman-active tag unattached to a target and at least one superparamagnetic Raman-active complex. The Raman-active complex includes a Raman-active tag attached to a target. The Raman-active tag includes a Raman-active particle and a target-binding moiety comprising an antibody.

The accompanying figures, which are incorporated in and constitute part of this specification, are included to illustrate and provide a further understanding of the method and complex according to embodiments of the invention. Together with the description, the drawings serve to explain the principles of the invention.

### BRIEF DESCRIPTION OF THE DRAWINGS

FIG. 1 is a schematic representation of a known unattached Raman-active tag;

FIG. 2A is a schematic representation of a known Raman-active complex;

FIG. 2B is another schematic representation of a known Raman-active complex;

FIG. 3 is a schematic representation of a superparamagnetic Raman-active complex in accordance with an embodiment of the invention;

FIG. 4 is a schematic representation of a method of separating a Raman-active tag unattached to a target from a Raman-active complex in accordance with an embodiment of the invention;

FIG. 5 is another schematic representation of a method of separating a Raman-active tag unattached to a target from a Raman-active complex in accordance with an embodiment of the invention; and

FIG. 6 is a flow chart of a method of separating a Raman-active tag unattached to a target from a Raman-active complex in accordance with an embodiment of the invention.

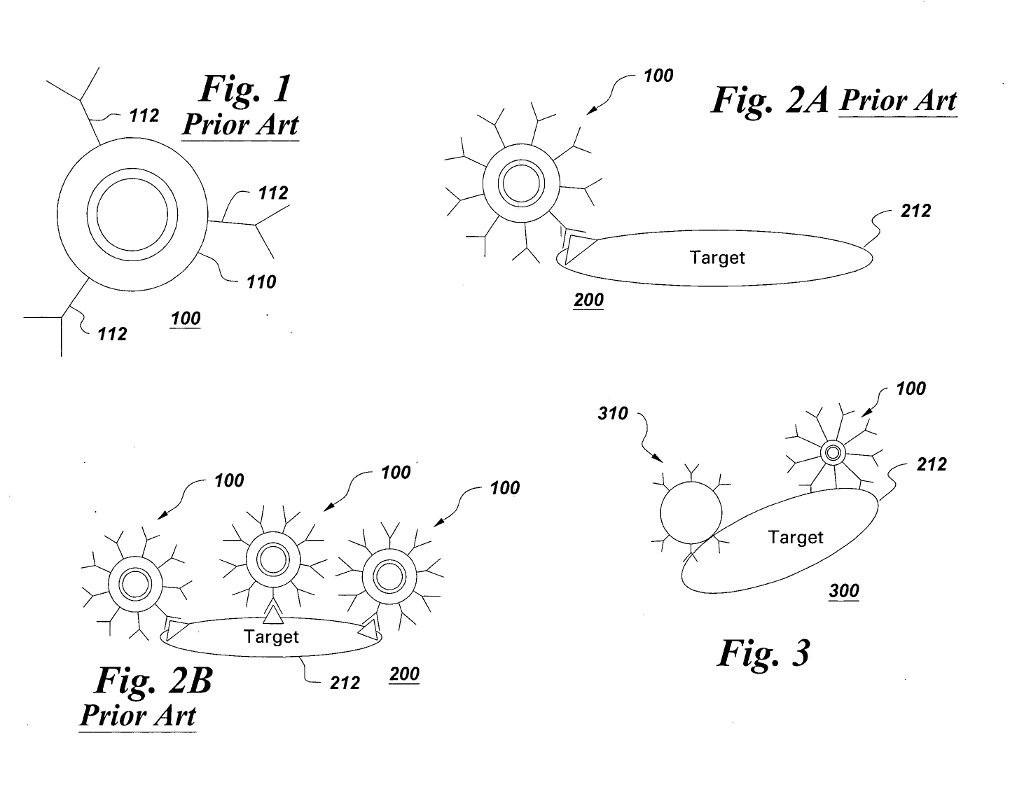

### DETAILED DESCRIPTION

Referring to the drawings in general, it will be understood that the illustrations are for the purpose of describing a particular embodiment of the invention and are not intended to limit the invention thereto.

Whenever a particular embodiment of the invention is said to comprise or consist of at least one element of a group and combinations thereof, it is understood that the embodiment may comprise or consist of any of the elements of the group, either individually or in combination with any of the other elements of that group. Furthermore, when any variable occurs more than one time in any constituent or in formula, its definition on each occurrence is independent of its definition at every other occurrence. Also, combinations of substituents and/or variables are permissible only if such combinations result in stable Raman-active tags or Raman-active complexes.

With reference to FIG. 3, there is shown one embodiment of a superparamagnetic Raman-active complex 300. The superparamagnetic Raman-active complex 300 includes one or more Raman-active tags 100 attached to a target 212 and one or more superparamagnetic particles 310 attached to the target 212 or the Raman-active tag 100. Unless noted otherwise, the word “Raman” and “Raman-active” includes Raman, surface-enhanced Raman, resonance Raman, and surface-enhanced resonance Raman spectroscopies.

Examples of superparamagnetic particles include, but are not limited to, nano or micron sized beads that are attracted by a magnetic field but retain little or no residual magnetism when the field is removed. In one embodiment, the superparamagnetic particles are capable of responding to a magnetic field but are not magnetic. Examples of superparamagnetic particles include, but are not limited, iron oxides such as magnetite. The superparamagnetic particles may be formed to have a predetermined shape and/or size, such as but are not limited to, nano or micron sized and bead shaped, based on the end-use for the particles.

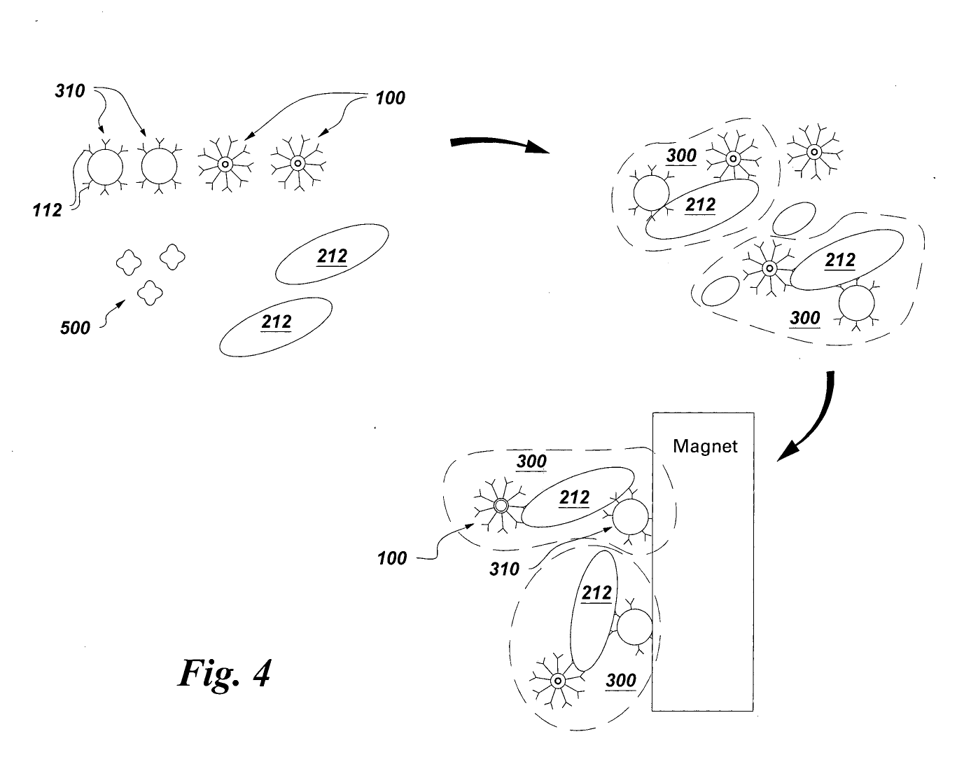

With reference to FIG. 4-FIG. 6, methods of applying a magnetic field to a mixture that includes one or more Raman-active tag unattached to a target and one or more superparamagnetic Raman-active complexes are described. The Raman-active tag is unattached to a target while the Raman-active complex is attached to target.

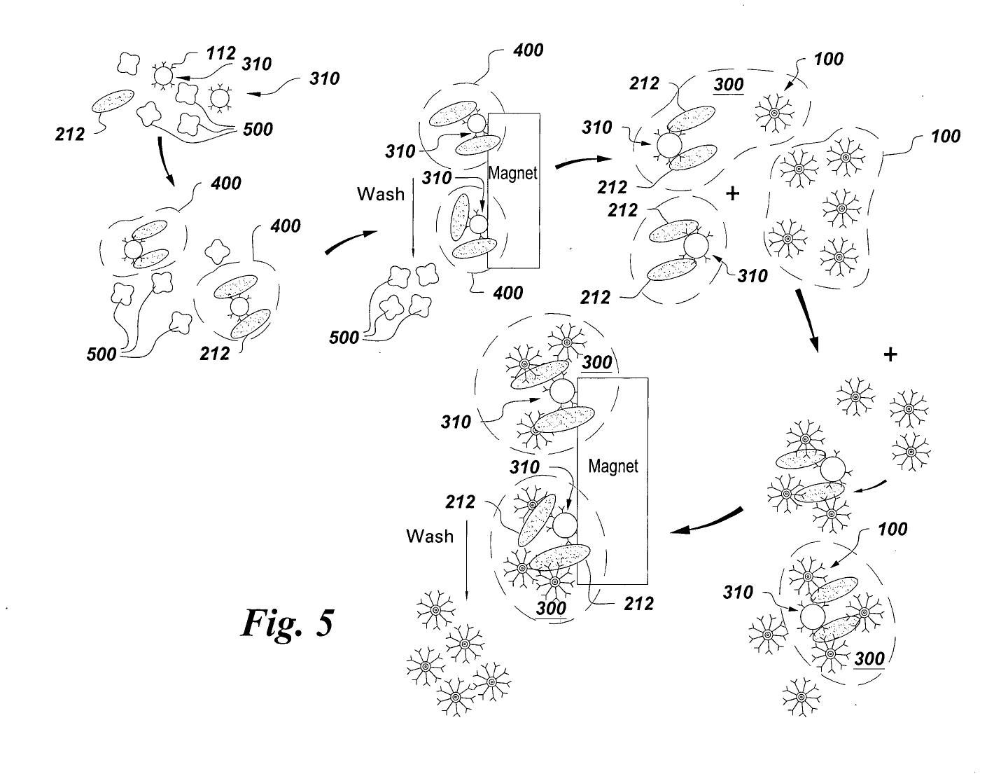

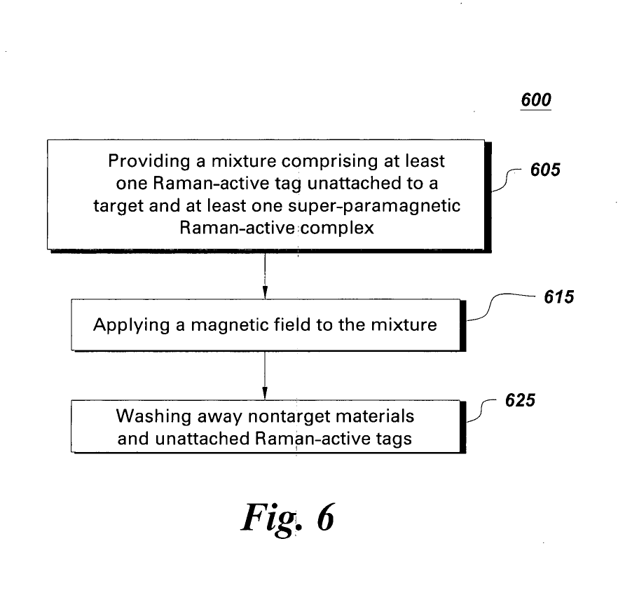

FIGS. 4 and 5 are schematic representations of methods of separating one or more Raman-active tags unattached to a target from one or more Raman-active complexes. FIG. 6 is a flow chart of an embodiment of a method of separating one or more Raman-active tags from one or more Raman-active complexes.

As described in FIG. 6, the method includes, at Step 605, of providing a mixture having one or more Raman-active tags and or one or more superparamagnetic Raman-active complexes as described above. The mixture may also include other non-target components (500), such as impurities, toxins, and the like, as shown in FIG. 4 and FIG. 5.

The Raman-active tags and superparamagnetic Raman-active complexes may be provided in a manner consistent with the end-use of the complexes. In one embodiment, one or more superparamagnetic particles 310, one or more Raman-active tags 100, and one or more targets 212 are combined to form a superparamagnetic Raman-active complex 300. The superparamagnetic particles 310, Raman-active tags 100, and targets 212 may be provided simultaneously, as in FIG. 4, or sequentially relative to each other as in FIG. 5. Furthermore, the superparamagnetic particles 310, Raman-active tags 100, and targets 212 may be sequentially provided in any permutation relative to each other. For example, in one embodiment, the superparamagnetic particles 310 and the targets 212 are provided before the Raman-active tags 100 as in FIG. 5. In another embodiment, the Raman-active tags 100 and targets 212 may be provided before the superparamagnetic particles 310. The superparamagnetic particles 310 may attach to Raman-active complexes 200 to form the superparamagnetic Raman-active complexes 300 as described above. The superparamagnetic particles 310 and the target may attach to each other to form a superparamagnetic-target complex 400.

The superparamagnetic particles 310, Raman-active tags 100, and targets 212 may attach via a predetermined attachment mechanism and at a predetermined site of attachment. In one embodiment, the superparamagnetic particles 310, Raman-active tags 100, and targets 212 attach together to form the superparamagnetic Raman-active complexes 300. In another embodiment, the superparamagnetic particles 310 and targets 212 attach together to form the superparamagnetic-target complex 400. Examples of attaching include, but are not restricted to, electrostatically, chemically, and physically, as well as covalent and non-covalent attachment. Attached also includes at least partially attached. Approximating language, as used herein throughout the specification and claims, may be applied to modify any quantitative or qualitative representation that could permissibly vary without resulting in a change in the basic function to which it is related. Attached particles may include those particles that are only partially attached, or are temporarily attached to each other. Furthermore, the superparamagnetic particles, Raman-active tags, and targets may attach at a plurality of sites to form a superparamagnetic Raman-active complex 300. The superparamagnetic particles, and targets may attach at a plurality of sites to form a superparamagnetic-target complex 400. In differing embodiments, each of the attachment sites may be by a different mode of attachment.

Step 615 includes applying a magnetic field to the mixture. The magnetic field may attract and immobilize the superparamagnetic particles 310 as well as the superparamagnetic Raman-active complex 300 which include the superparamagnetic particles, the target, and Raman-active tags. Immobilized means at least partially immobilized such that the superparamagnetic particles superparamagnetic Raman-active complex 300 Approximating language, as used herein throughout the specification and claims, may be applied to modify any quantitative or qualitative representation that could permissibly vary without resulting in a change in the basic function to which it is related. Accordingly, “immobilized” may be used in combination with a term, and may include an insubstantial amount of mobility while still being considered immobilized. The strength and duration of the magnetic field, as well as the magnetic material, may be varied based on desired end result. In one embodiment, the magnetic field strength is in a range from about 1 gauss to about 2000 gauss. In a particular embodiment, the magnetic field strength is in a range from about 50 to about 500 gauss. The duration of the magnetic field may last from about 1 second to about 5 minutes. In a particular embodiment, the duration of the magnetic field may last for about 1 minute. In one embodiment, a bar magnet may be used to apply the magnetic field.

Particles that are subject to manipulation via magnetic field may include superparamagnetic particles having an average particle size in a range of from about 10 nanometers (nm) to about 10 micrometers. In another embodiment, the superparamagnetic particles may have an average particle size in a range of from about 0.3 micrometers to about 1.5 micrometers.

Step 605 of providing a mixture and Step 615 of applying a magnetic field may occur sequentially or simultaneously.

A Raman spectrum of the superparamagnetic Raman-active complex may be taken. The Raman spectrum may be taken directly after a washing Step 625. The washing step may remove some or all unattached Raman-active tags and other non-target components that are in solution. The superparamagnetic Raman-active complexes are then removed from the magnetic field and resuspended in a small volume of buffer to take the Raman spectrum.

The Raman spectrum may be correlated to the presence of a target attached to the Raman-active complex. The correlation of the Raman spectrum may lead to the identification and/or quantification of the target attached to the Raman-active complex.

A mixture may include a plurality of targets and the method may detect the plurality of targets sequentially or simultaneously relative to each other. Thus, in one embodiment, the plurality of Raman-active complexes are attached to a plurality of targets. The method may further include generating a plurality of Raman spectra. The plurality of Raman spectrums may be correlated to the presence of a plurality or targets that are different from each other. Detection, identification, and or quantification of the plurality of the targets may also be based on correlating the plurality of Raman signals to the plurality of targets in the mixture.

Raman-Active Tag and Raman-Active Particle

In one embodiment, the Raman-active tag is immuno-functionalized. Immuno-functionalized Raman-active tags detect the presence of one or more targets that are pathogenic organisms or other materials. Immuno-functionalized Raman-active tags include Raman-active tags attached to one or more target-binding moieties that are antibodies. The target-binding moiety is capable of attaching to a target. In one embodiment the target-binding moiety allows the Raman-active tag to attach to a target to form a Raman-active complex.

Examples of other target-binding moieties include, but are not limited to, antibodies, aptamers, polypeptides, nucleic acid, peptide nucleic acids, avidin, streptavidin, and derivatives of avidin and streptavidin. The Raman-active tag may include one target-binding moiety or a plurality of target-binding moieties. The plurality of target-binding moieties may all be of the same kind of target-binding moieties or different kinds of target-binding moieties.

The Raman-active tag includes a Raman-active particle attached to one or more target-binding moieties. The Raman-active particles may be of various predetermined sizes, shapes, and materials. In one embodiment, the Raman-active particle includes a core, a coating, and a Raman-active analyte. One or more cores, coatings, and analytes may be included within the Raman-active particle. The analyte is at least partially within the coating and the coating at least partially covers the core. In a particular embodiment, the coating covers the core.

In one embodiment, the core has a metallic surface. The core may include a metal such as, but not limited to, Au, Ag, Cu, Ni, Pd, Pt, Na, Al, and Cr, either individually or through a combination of two or more thereof. The core may include other inorganic or organic materials, provided the surface of the core is metallic. In a particular embodiment, the core includes Au.

The shape of the core may be selected with reference to a particular desired effect. For example, the core may be in the shape of a sphere, fiber, plate, cube, tripod, pyramid, rod, tetrapod, or any non-spherical object. In one embodiment, the core is spherical.

The size of the core may be selected with regard to the particles composition and intended use. In one embodiment, the cores have an average diameter in a range from about 1 nm to about 500 nm. In another embodiment, the cores have an average diameter less than about 100 nm. In yet another embodiment, the cores have an average diameter in a range from about 12 nm to about 100 nm.

In one embodiment, the coating includes a stabilizer to reduce or eliminate the Raman-active particle aggregation. The coating stabilizes the Raman-active particle in one way by inhibiting aggregation of Raman-active particles. The coating is sufficiently thick to stabilize the Raman-active particle. In one embodiment, the coating has a thickness in a range from about 1 nm to about 500 nm. In another embodiment, the coating has a thickness in a range from about 5 nm to about 30 nm.

In one embodiment, the coating includes an elemental oxide. In a particular embodiment, the coating includes silicon. The percentage of silicon may depend on one or more factors. Such factors may include the intended use of the Raman-active particle, the composition of the core, the degree to which the coating is to be functionalized, the desired density of the coating for a given application, the desired melting point for the coating, the identity of any other materials which constitute the coating, and the technique by which the Raman-active particle is to be prepared. In one embodiment, the element in the elemental oxide of the coating includes at least about 50-mole % silicon. In another embodiment, the element in the elemental oxide of the coating includes at least about 70-mole %. Yet, in another embodiment, the element in the elemental oxide of the coating includes substantially silicon.

In yet another embodiment, the coating includes a composite. A composite coating may include oxides of one or more elements such as, but not limited to, Si, B, Al, Ga, In, Sc, Y, La, Ti, Zr, Hf, V, Nb, Ta, Cr, Mn, Fe, Co, Ni, Li, Na, K, Rb, Cs, Be, Mg, Ca, Sr, Ba, Zn, Cd, Ge, Sn, and Pb. Furthermore, the coating may have one or more sub-layers to form a multi-layer coating. Each of the coating layers in the multilayer coating individually may include differing coating compositions, such as 50-mole % silicon oxide in one coating layer and a composite coating in another coating layer.

The Raman-active particle includes one or more Raman-active analytes. In one embodiment, the Raman-active analyte exhibits Raman scattering when in the vicinity of a metallic core or a metallic surface of a core. Examples of Raman-active analytes include, but are not limited to, 4-mercaptopyridine, 2-mercaptopyridine (MP), trans-bis(pyridyl)ethylene (BPE), naphthalene thiol (NT), 4,4′-dipyridyl (DPY), quinoline thiol (QSH), and mercaptobenzoic acid, either individually or a combination of two or more thereof. In a particular embodiment, the Raman-active analyte includes trans-bis(pyridyl)ethylene and or quinoline thiol.

In one embodiment, the Raman-active analyte is at least partially disposed within the coating. The Raman-active analyte can be at least partially within the coating in various orientations, such as, but not limited to, dispersed within the coating, within and around the coating, or embedded within the coating. Furthermore, a plurality of analytes may be within the coating. The plurality of analytes may be within the coating at a plurality of sites or at a single site. The analytes may be within the coating by a different mode, such as dispersed within the coating, around the coating, or embedded within the coating.

The Raman-active particle may include one core within a coating or multiple cores within a coating. The multiple cores are non-aggregated or closer together. The selection as to how many cores should be contained within a coating may depend on the particular application for which the Raman-active particles are being used. Adjusting process conditions may obtain Raman-active particles with a single core contained in the coating. For example, the coating may also stabilize a core against aggregating with another core.

The Raman-active particle may differ in shape and size from application to application. In one embodiment, the Raman-active particles are substantially spherical and have an average diameter in a range less than about 1000 nm. In a particular embodiment, the Raman-active particle has an average diameter less than about 100 nm

In one embodiment, the Raman-active particle includes one or more linkers. The linker binds to the core and interacts with a surface of the coating. The linker allows or facilitates the coating to attach to the surface of the core. The linker may be a molecule having a functional group. The functional group can bind to the metal surface of the core and bind to the coating. An example of a linker is an alkoxysilane. Examples of alkoxysilanes include trialkoxysilanes. Trialkoxysilane linkers may be used to deposit coatings comprising silica. Suitable trialkoxysilane linkers include, but are not limited to, aminopropyl trimethoxysilane (APTMS), aminopropyl triethoxysilane, mercaptopropyl trimethoxysilane, mercaptopropyl triethoxysilane, hydroxypropyl trimethoxysilane, and hydroxypropyl triethoxysilane, either individually or in combinations of two or more thereof.

When more than one analyte, coating, linker, and core are present, the definition on each occurrence is independent of the definition at every other occurrence. Also, combinations of an analyte, coating, linker, and core are permissible if such combinations result in stable Raman-active particles. Also, methods in combining an analyte, coating, linker, and core are permissible if such combinations result in stable Raman-active particles.

Targets and Target-Binding Moieties

Target-binding moieties may attach to the target, directly or indirectly. Examples of attaching include, but are not restricted to, electrostatically, chemically, and physically. Examples of target-binding moieties include, but are not limited to, antibodies, aptamers, polypeptides, peptides, nucleic acids, avidin, streptavidin, and derivatives of avidin and streptavidin, either individually or in any combination thereof. The Raman-active tag may include one target-binding moiety or a plurality of target-binding moieties. Furthermore, the plurality of target-binding moieties may be of the same or similar kind capable of attaching to the same type of targets. The plurality of target-binding moieties may also be of differing kinds capable of attaching to different types of target. Detection of the plurality of the targets is based on the presence of Raman signal after removing any Raman-active tags that are unattached to a target from the test mixture.

Other non-limiting examples of target-binding moieties include, but are not limited to, proteins, peptides, polypeptides, glycoproteins, selected ligands, lipoproteins, phospholipids, oligonucleotides, or the like, e.g. enzymes, immune modulators, receptor proteins, antibodies and antibody fragments, which preferentially bind marker substances that are produced by or associated with the target site.

Proteins are known that preferentially bind marker substances that are produced by or associated with lesions. For example, antibodies can be used against cancer-associated substances, as well as against any pathological lesion that shows an increased or unique antigenic marker, such as against substances associated with cardiovascular lesions, for example, vascular clots including thrombi and emboli, myocardial infarctions and other organ infarcts, and atherosclerotic plaques; inflammatory lesions; and infectious and parasitic agents.

Cancer states include carcinomas, melanomas, sarcomas, neuroblastomas, leukemias, lymphomas, gliomas, myelomas, and neural tumors. Infectious diseases include those caused by body invading microbes or parasites.

The protein substances useful as target-binding moieties include protein, peptide, polypeptide, glycoprotein, lipoprotein, or the like; e.g. hormones, lymphokines, growth factors, albumin, cytokines, enzymes, immune modulators, receptor proteins, antibodies and antibody fragments. The protein substances of particular interest are antibodies and antibody fragments. The terms “antibodies” and “antibody fragments” mean generally immunoglobulins or fragments thereof that specifically bind to antigens to form immune complexes.

The antibody may be a whole immunoglobulin of any class; e.g., IgG, IgM, IgA, IgD, IgE, chimeric or hybrid antibodies with dual or multiple antigen or epitope specificities. It can be a polyclonal antibody, particularly a humanized or an affinity-purified antibody from a human. It can be an antibody from an appropriate animal; e.g., a primate, goat, rabbit, mouse, or the like. If a paratope region is obtained from a non-human species, the target may be humanized to reduce immunogenicity of the non-human antibodies, for use in human diagnostic or therapeutic applications. Such a humanized antibody or fragment thereof is termed “chimeric.” For example, a chimeric antibody comprises non-human (such as murine) variable regions and human constant regions. A chimeric antibody fragment can comprise a variable binding sequence or complementarity-determining regions (“CDR”) derived from a non-human antibody within a human variable region framework domain. Monoclonal antibodies are also suitable because of their high specificities. Useful antibody fragments include F(ab')₂, F(ab)₂, Fab', Fab, Fv, and the like including hybrid fragments. Particular fragments are Fab', F(abF')₂, Fab, and F(ab)₂. Also useful are any subfragments retaining the hypervariable, antigen-binding region of an immunoglobulin and having a size similar to or smaller than a Fab' fragment. An antibody fragment can include genetically engineered and/or recombinant proteins, whether single-chain or multiple-chain, which incorporate an antigen-binding site and otherwise function in vivo as immobilized target-binding moieties in substantially the same way as natural immunoglobulin fragments. The fragments may also be produced by genetic engineering.

Examples of selective ligands include porphyrins, ethylenediaminetetraacetic acid (EDTA), and zinc fingers. Selective ligand means a ligand selective for a particular target or targets.

Mixtures of antibodies and immunoglobulin classes can be used, as can hybrid antibodies. Multispecific, including bispecific and hybrid, antibodies and antibody fragments are sometimes desirable for detecting and treating lesions and include at least two different substantially monospecific antibodies or antibody fragments, wherein at least two of the antibodies or antibody fragments specifically bind to at least two different antigens produced or associated with the targeted lesion or at least two different epitopes or molecules of a marker substance produced or associated with the targeted lesion. Multispecific antibodies and antibody fragments with dual specificities can be prepared analogously to anti-tumor marker hybrids.

Suitable MAbs against microorganisms (bacteria, viruses, protozoa, other parasites) responsible for the majority of infections in humans may be used for in vitro diagnostic purposes. These antibodies, and newer MAbs, are also appropriate for use.

Proteins useful for detecting and/or treating cardiovascular lesions include fibrin-specific proteins; for example, fibrinogen, soluble fibrin, antifibrin antibodies and fragments, fragment E₁ (a 60 kDa fragment of human fibrin made by controlled plasmin digestion of crosslinked fibrin), plasmin (an enzyme in the blood responsible for the dissolution of fresh thrombi), plasminogen activators (e.g., urokinase, streptokinase and tissue plasminogen activator), heparin, and fibronectin (an adhesive plasma glycoprotein of 450 kDa) and platelet-directed proteins; for example, platelets, antiplatelet antibodies, and antibody fragments, anti-activated platelet antibodies, and anti-activated platelet factors.

In one embodiment, the target-binding moiety includes a MAb or a fragment thereof that recognizes and binds to a heptapeptide of the amino terminus of the β-chain of fibrin monomer. Fibrin monomers are produced when thrombin cleaves two pairs of small peptides from fibrinogen. Fibrin monomers spontaneously aggregate into an insoluble gel, which is further stabilized to produce blood clots.

The disclosure of various antigens or biomarkers that can be used to raise specific antibodies against them (and from which antibodies fragments may be prepared) serves only as examples, and is not to be construed in any way as a limitation of the invention.

Targets

Targets include living targets and non-living targets. Examples of targets include, but are not limited to, prokaryotic cells, eukaryotic cells, bacteria, viruses, proteins, polypeptides, toxins, liposomes, beads, ligands, amino acids, and nucleic acids, either individually or in any combinations thereof. The target includes extracts of the above living or non-living targets.

Examples of prokaryotic cells include, but are not limited to, bacteria also include extracts thereof. Examples of eukaryotic cells include, but are not limited to, yeast cells, animal cells and tissues. Examples of toxins include, but are not limited to, anthrax. Examples of beads include, but are not limited to, latex, polystyrene, silica and plastic.

The term “peptide” refers to oligomers or polymers of any length wherein the constituent monomers are alpha amino acids linked through amide bonds, and encompasses amino acid dimers as well as polypeptides, peptide fragments, peptide analogs, naturally occurring proteins, mutated, variant or chemically modified proteins, fusion proteins, and the like. The amino acids of the peptide molecules may be any of the twenty conventional amino acids, stereoisomers (e.g., D-amino acids) of the conventional amino acids, structural variants of the conventional amino acids, e.g., iso-valine, or non-naturally occurring amino acids such as α,α-disubstituted amino acids, N-alkyl amino acids, β-alanine, naphthylalanine, 3-pyridylalanine, 4-hydroxyproline, O-phosphoserine, N-acetylserine, N-formylmethionine, 3-methylhistidine, 5-hydroxylysine, and nor-leucine. In addition, the term “peptide” encompasses peptides with posttranslational modifications such as glycosylations, acetylations, phosphorylations, and the like.

The term “oligonucleotide” is used herein to include a polymeric form of nucleotides of any length, either ribonucleotides or deoxyribonucleotides. This term refers only to the primary structure of the molecule. Thus, the term includes triple-, double-and single-stranded DNA, as well as triple-, double-and single-stranded RNA. The term also includes modifications, such as by methylation and/or by capping, and unmodified forms of the oligonucleotide. More particularly, the term includes polydeoxyribonucleotides (containing 2-deoxy-D-ribose), polyribonucleotides (containing D-ribose), any other type of polynucleotide which is an N-glycoside or C-glycoside of a purine or pyrimidine base, and other polymers containing nonnucleotidic backbones, for example, polyamide (e.g., peptide nucleic acids (PNAs)) and polymorpholine (commercially available from the Anti-Virals, Inc., Corvallis, Oreg., as Neugene) polymers, and other synthetic sequence-specific nucleic acid polymers, providing that the polymers contain nucleobases in a configuration that allows for base pairing and base stacking, such as is found in DNA and RNA. There is no intended distinction in length between the terms “polynucleotide”, “oligonucleotide”, “nucleic acid” and “nucleic acid molecule”, and these terms refer only to the primary structure of the molecule. Thus, these terms include, for example, 3′-deoxy-2′,5′-DNA, oligodeoxyribonucleotide N3′P5′ phosphoramidates, 2′-O-alkyl-substituted RNA, double- and single-stranded DNA, as well as double-and single-stranded RNA, DNA:RNA hybrids, and hybrids between PNAs and DNA or RNA, and also include known types of modifications, for example, labels which are known in the art, methylation, “caps”, substitution of one or more of the naturally occurring nucleotides with an analog, internucleotide modifications such as, for, example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc. ), those containing alkylators, those with modified linkages (e.g., alpha anomeric nucleic acids, etc.), as well as unmodified forms of the polynucleotide or oligonucleotide. The term also includes other kinds of nucleic acids such as, but not limited to, locked nucleic acids (LNAs).

The terms “nucleoside” and “nucleotide” also include those moieties that contain not only the known purine and pyrimidine bases, but also other heterocyclic bases, which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

Furthermore, modifications to nucleotidic units include rearranging, appending, substituting for or otherwise altering functional groups on the purine or pyrimidine base that form hydrogen bonds to a respective complementary pyrimidine or purine. The resultant modified nucleotidic unit optionally may form a base pair with other such modified nucleotidic units but not with A, T, C, G or U. Basic sites may be incorporated which do not prevent the function of the polynucleotide. Some or all of the residues in the polynucleotide optionally can be modified in one or more ways.

The term “antibody” as used herein includes antibodies obtained from both polyclonal and monoclonal preparations, as well as hybrid (chimeric) antibody molecules; F(ab')2 and F(ab) fragments; Fv molecules (noncovalent heterodimers); single-chain Fv molecules (sFv); dimeric and trimeric antibody fragment constructs; humanized antibody molecules; and any functional fragments obtained from such molecules, wherein such fragments retain specific-binding properties of the parent antibody molecule. In one embodiment, the target is attached to one Raman-active complex or a plurality of Raman-active complexes.

The following example illustrates the features of the invention and is not intended to limit the invention thereto.

### MAGNETIC PARTICLE METHOD EXAMPLE

### Generalized

An amount of target microorganisms 212, which includes but is not restricted to bacteria, spores, and viruses, is added to a sample container such as an eppindorf tube.

A quantity of nanometer or micrometer sized superparamagnetic (SPR) particles 310 attached to antibodies against the target microorganism 212 are added to the sample.

A quantity of Raman-active tags 100 attached to antibodies against the target microorganism 212 is added to the sample.

The sample is mixed and incubated at room temperature in a container, such as an eppendorf tube, for a period of time.

The mixture is placed in a magnetic field. The magnetic field immobilizes one or more SPR particles 310, as well as one or more superparamagnetic Raman-active complex 300 which includes one or more SPR particles 310. The magnetic field immobilizes the SPR particles and the superparamagnetic Raman-active complex 300 onto the wall of the eppendorf tube.

Unattached Raman-active tags and other components of the mixture remain in solution and are removed by washing.

After washing, the magnetic field is removed and the superparagmagnetic Raman-active complexes 300 (i.e. SPR Raman-active complexes) are resuspended in a small volume of buffer.

A portion of the buffer is then analyzed for the presence of a Raman-active signal.

Thus, Example 1 demonstrates the use of immuno-functionalized Raman-active tags 100 to detect the presence of a specific target organism 212. In these experiments, a Raman signal only is detected when the appropriate target organism 212 and Raman-active tags 100 immuno-functionalized for that specific target organism 212 to detect the presence of that specific target organism 212 are both present.

While the invention has been described in detail in connection with only a limited number of aspects, it should be readily understood that the invention is not limited to such disclosed aspects. Rather, the invention can be modified to incorporate any number of variations, alterations, substitutions or equivalent arrangements not heretofore described, but which are commensurate with the scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A method comprising:
applying a magnetic field to a mixture comprising at least one Raman-active active tag unattached to a target and at least one superparamagnetic Raman-active complex that comprises a target.

2. The method of claim 1, wherein the superparamagnetic Raman-active complex comprises a superparamagnetic particle, a Raman-active tag, and a target.

3. The method of claim 2, wherein the superparamagnetic particle has an average diameter that is in a range of from about 10 nanometers to about 10 micrometers.

4. The method of claim 2, wherein the superparamagnetic particle has an average diameter that is in a range of from about 0.3 micrometers to about 1.5 micrometers

5. The method of claim 1, further comprising forming a superparamagnetic Raman-active complex by providing a superparamagnetic particle, a target, and a Raman-active tag simultaneously with each other.

6. The method of claim 1, further comprising forming a superparamagnetic Raman-active complex by providing a superparamagnetic particle, a Raman-active tag, and target sequentially relative to each other.

7. The method of claim 6, further comprising forming a superparamagnetic Raman-active complex by providing a superparamagnetic particle and a Raman-active tag before the target.

8. The method of claim 6, further comprising forming a superparamagnetic Raman-active complex by providing a superparamagnetic particle and a target before the Raman-active tag.

9. The method of claim 1, wherein applying the magnetic field comprises applying a sufficient magnetic field to separate the at least one Raman-active tag unattached to a target from the at least one superparamagnetic Raman-active complex.

10. The method of claim 1, wherein the Raman-active tag comprises a Raman-active particle and a target-binding moiety, wherein the target-binding moiety is capable of attaching to a target.

11. The method of claim 10, wherein the target-binding moiety comprises at least one chemical moiety selected from a group consisting of antibodies, aptamers, nucleic acids, and polypeptides.

12. The method of claim 1, further comprising generating a Raman spectrum of the superparamagnetic Raman-active complex subsequent to applying the magnetic field.

13. The method of claim 12, further comprising correlating the generated Raman spectrum to a presence of the target.

14. The method of claim 12, further comprising correlating the generated Raman spectrum to an identification of the target.

15. The method of claim 12, further comprising correlating the generated Raman spectrum to a quantity of the target.

16. The method of claim 1, wherein a plurality of superparamagnetic Raman-active complexes attach to a plurality of targets.

17. The method of claim 16, further comprising generating a plurality of the Raman spectrums, wherein the plurality of Raman spectrums correlate to the presence of a plurality of targets that are different from each other.

18. The method of claim 16, further comprising correlating the plurality of the generated Raman spectrums to an identification of the plurality of targets that are different from each other.

19. The method of claim 16, further comprising correlating the plurality of the generated Raman spectrums to a quantification of the plurality of targets that are different from each other.

20. The method of claim 1, wherein the target comprises at least one target selected from a group consisting of prokaryotic cells, eukaryotic cells, viruses, bacteria, proteins, polypeptides, toxins, liposomes, nucleic acids, spores, and beads.

21. The method of claim 1, wherein the target is attached to a plurality of Raman-active complexes.

22. A method comprising:
applying a magnetic field to a mixture comprising at least one Raman-active tag unattached to a target and at least one superparamagnetic Raman-active complex, wherein the Raman-active tag comprises a Raman-active particle and a target-binding moiety comprising an antibody.

23. A superparamagnetic Raman-active complex comprising:
a Raman-active tag attached to a target; and
a superparamagnetic particle attached to the target or the Raman-active tag.
